(19) United States
(12) Patent Application Publication
(10) Pub. No.: US 2020/0108179 A1
(43) Pub. Date: Apr. 9, 2020

(54) BONE SUBSTITUTE AND METHOD FOR PRODUCING BONE SUBSTITUTE

(71) Applicant: OLYMPUS CORPORATION, Tokyo (JP)

(72) Inventors: Masaki HAYASHI, Tokyo (JP); Jun HONDA, Tokyo (JP)

(73) Assignee: OLYMPUS CORPORATION, Tokyo (JP)

(21) Appl. No.: 16/699,230

(22) Filed: Nov. 29, 2019

Related U.S. Application Data
(63) Continuation of application No. PCT/JP2017/020688, filed on Jun. 2, 2017.

(51) Int. Cl. A61L 27/54; A61L 27/12; A61L 27/32; A61L 27/30; A61L 27/10; A61L 27/56

(52) U.S. Cl. CPC A61L 27/54; A61L 27/12; A61L 27/32; A61L 2420/02; A61L 27/10; A61L 27/56; A61L 2430/02; A61L 27/306

(57) ABSTRACT

This bone substitute includes a calcium phosphate-type porous ceramic body having spherical voids; and a bioactive glass layer that is formed of a bioactive glass, coats an inner surface of each of the voids and has a thickness of less than 50 μm. The porous body has communication holes through which the voids communicate with each other.

The present invention relates to a bone substitute and a method for producing a bone substitute. This application is a continuation application based on PCT Patent Application No.PCT/JP2017/020688, filed Jun. 2, 2017, the content of which is incorporated herein by reference.

### BACKGROUND

In the medical field, treatment for restoring a bone defect part, which is caused by removal of a benign bone tumor or an injury such as fracture, or caused by spinal surgery or artificial joint surgery, has been performed. In the treatment, the bone defect part is restored by filling a bone substitute to the bone defect part and allowing the bone to regenerate. In addition, when there is varus deformity of a lower limb, high tibial osteotomy (HTO) in which a part of tibia is excised or incised, and a bone substitute is filled into the excised or incised part to correct the part is performed. Hydroxyapatite (HAP) and calcium phosphate are known as bone substitutes used for such treatment.

As a bone substitute, for example, a porous calcium phosphate material such as β-tricalcium phosphate (β-TCP) may be used for the purpose of preventing foreign matter from remaining in the body. When β-TCP remains in contact with a bone defect part, osteoclasts absorb β-TCP, and osteoblasts form new bone. That is, the bone substitute which is filled in a bone defect part is replaced with new bone over time.

In treatment using a bone substitute, it is desired to shorten a period until the bone substitute is replaced with new bone. As a method for shortening the period during which the bone substitute is replaced with new bone, there is a method in which a contact area between bone cells and a porous calcium phosphate substance is increased to improve the amount able to be absorbed by bone cells. To this end, a porosity of the porous calcium phosphate substance needs be increased to improve the resorption by bone cells, and also voids of the porous calcium phosphate body need to have a sufficient size for smoothly supplying nutrients and discharging waste. However, a load resistance strength of the porous calcium phosphate body decreases when the porosity is increased by providing large-diameter voids. The bone substitute may be compressed when the bone substitute is loaded into the bone defect part. In addition, after the bone substitute is implanted, the bone substitute is subjected to a load from the bones in the vicinity of the implanted site according to a motion of a patient. When the compressive strength of the bone substitute after the implantation is low, the patient is restricted from moving the implantation site of the bone substitute until new bone is formed, and is burdened. Therefore, the bone substitute is required to have sufficient strength to withstand the pressure during the implantation and the pressure until new bone is formed after the implantation. Thus, for example, in a bone substitute disclosed in Japanese Unexamined Patent Application, First Publication No. 2006-68249, a bioactive glass is adhered to inner surfaces of voids of a porous ceramic body.

### SUMMARY

A bone substitute according to a first aspect of the present disclosure includes a calcium phosphate-type porous ceramic body having spherical voids; and a bioactive glass layer which is formed of a bioactive glass, coats an inner surface of each of the voids and has a thickness of less than 50 μm, wherein the porous body has communication holes through which the voids communicate with each other.

According to a second aspect of the present disclosure, in the bone substitute according to the first aspect, the thickness of the bioactive glass layer may be 10 μm or more and 20 μm or less.

According to a third aspect of the present disclosure, in the bone substitute according to the first aspect, in the calcium phosphate-type porous ceramic body, a content of the bioactive glass in a portion of each of the voids other than the inner surface may be less than 5% by mass when a mass of the bone substitute is 100.

According to a fourth aspect of the present disclosure, in the bone substitute according to the first aspect, an average diameter of the voids of the calcium phosphate-type porous ceramic body may be 150 μm or more.

According to a fifth aspect of the present disclosure, in the bone substitute according to the first aspect, an average diameter of the voids of the calcium phosphate-type porous ceramic body may be 200 μm or more and 500 μm or less.

According to a sixth aspect of the present disclosure, in the bone substitute according to the first aspect, a porosity of the calcium phosphate-type porous ceramic body may be 65% or more and less than 90%.

According to a seventh aspect of the present disclosure, in the bone substitute according to the first aspect, the bioactive glass may include silicon dioxide, calcium oxide, sodium oxide, and diphosphorus pentoxide, and a composition ratio of the bioactive glass may be 45% by weight of silicon dioxide, 24.5% by weight of calcium oxide, 24.5% by weight of sodium oxide, and 6.0% by weight of diphosphorus pentoxide.

According to an eighth aspect of the present disclosure, in the bone substitute according to the first aspect, the communication holes may have two or more of the voids communicate with each other, and the communication holes may have a communication portion between the voids which has an opening dimension of 50 μm or more.

According to a ninth aspect of the present disclosure, a method for producing the bone substitute including a calcium phosphate-type porous ceramic body having spherical voids and a bioactive glass layer which is formed of a bioactive glass, coats an inner surface of each of the voids, the method including steps of: obtaining particles for forming holes by coating a fine powder of the bioactive glass on surfaces of core particles for forming holes; obtaining hollow particles of the bioactive glass by baking the particles for forming holes to remove the core particles; molding a bulk body from a slurry containing the hollow particles and calcium phosphate-type ceramic powder; and obtaining the bone substitute by baking the bulk body.

According to a tenth aspect of the present disclosure, a method for producing the bone substitute including a calcium phosphate-type porous ceramic body having spherical voids and a bioactive glass layer which is formed of a bioactive glass, coats an inner surface of each of the voids, the method including steps of obtaining particles for forming holes by coating fine powder of the bioactive glass on surfaces of hole forming core particles and obtaining particles for forming holes; molding a bulk body from a slurry containing the particles for forming holes and calcium phosphate-type ceramic powder; and obtaining the bone substitute by baking the bulk body to remove the core particles.

According to an eleventh aspect of the present disclosure, in the method for producing the bone substitute according to the ninth aspect, the core particles may have a spherical shape.

According to a twelfth aspect of the present disclosure, in the method for producing the bone substitute according to the ninth aspect, the core particles may be formed of a polymer.

According to a thirteenth aspect of the present disclosure, in the method for producing the bone substitute according to the tenth aspect, the core particles may have a spherical shape.

According to a fourteenth aspect of the present disclosure, in the method for producing the bone substitute according to the tenth aspect, the core particles may be formed of a polymer.

### BRIEF DESCRIPTION OF THE DRAWINGS

FIG. 1 is a schematic view showing a bone substitute according to an embodiment.

FIG. 2 is a schematic view showing a communication portion of voids in the bone substitute according to the embodiment.

FIG. 3 is a flowchart showing a method for manufacturing a bone substitute according to an embodiment.

FIG. 4 is a schematic view showing the method for producing the bone substitute according to the embodiment.

FIG. 5 is a flowchart showing a modified example of the method for producing the bone substitute according to the embodiment.

FIG. 6 is an SEM image of a bone substitute according to an example.

FIG. 7 is an SEM-EDX analysis image of the bone substitute according to the example.

FIG. 8A is a photograph taken at the time of a test in which the bone substitute of the example is immersed in rabbit blood.

FIG. 8B is a photograph taken at the time of a test in which the bone substitute of the example is immersed in rabbit blood.

FIG. 8C is a photograph taken at the time of the test in which the bone substitute of the example is immersed in the rabbit blood.

FIG. 8D is a photograph taken at the time of the test in which the bone substitute of the example is immersed in the rabbit blood.

FIG. 9 is an SEM image of a bone substitute according to a comparative example.

FIG. 10 is an SEM-EDX analysis image of the bone substitute according to the comparative example.

### DETAILED DESCRIPTION OF THE PREFERRED EMBODIMENTS

Hereinafter, an exemplary embodiment will be described with reference to the drawings.

FIG. 1 is a schematic view showing a bone substitute 1 according to an embodiment of the present disclosure. The bone substitute 1 according to the embodiment includes a calcium phosphate-type porous ceramic body 10 and a bioactive glass layer 20.

The calcium phosphate-type porous ceramic body is a porous body formed of a calcium phosphate-type ceramic and having a plurality of substantially spherical voids 11 formed therein. The calcium phosphate-type porous ceramic body is a base material for the bone substitute 1. Hereinafter, the calcium phosphate-type porous ceramic body is referred to as the “base material”.

Raw materials constituting the base material 10 include a calcium phosphate compound as a main component. The calcium phosphate compound is preferably any one of β-tricalcium phosphate (β-TCP), a-tricalcium phosphate (α-TCP), hydroxyapatite

(HAP), calcium deficient hydroxyapatite, fluoroapatite, tetracalcium phosphate, octacalcium phosphate (OCP), calcium carbonate, calcium hydrogen phosphate anhydrate, calcium hydrogen phosphate dihydrate, tetracalcium phosphate (TTCP), calcium diphosphate, calcium metaphosphate, and carbonate group-substituted hydroxyapatite (CHA). In the embodiment, β-TCP is used.

The base material 10 has an average diameter of the voids 11 of 150 μm or more. The average diameter of the voids 11 is an average value of diameters of portions excluding the bioactive glass layer which will be described later. When the average diameter of the voids 11 is 150 μm or more, the penetration ability of osteoblasts can be enhanced.

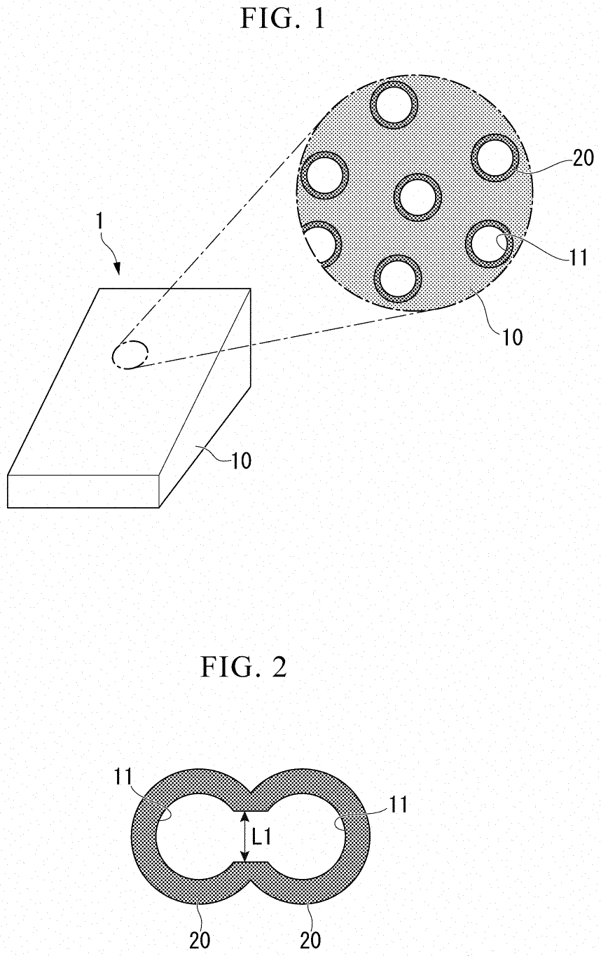

Furthermore, when a communication hole in which a plurality of voids 11 communicate with each other is formed, osteoblasts can easily penetrate into a deep portion of the base material 10, and a rate of replacement with new bone can be improved. At this time, when an opening dimension L1 (refer to FIG. 2) of a communication portion between the plurality of voids 11 is less than 50 μm, smooth circulation of osteoblasts cannot be expected. Therefore, the opening dimension L1 of the communication portion between the plurality of voids 11 is preferably 50 μm or more. When the average diameter of the voids 11 is less than 150 μm, it is difficult to set the opening dimension L1 of the communication portion between the plurality of voids 11 to 50 μm or more. The opening dimension L1 of the communication portion between the plurality of voids 11 can be measured by SEM image analysis.

An upper limit of the average diameter of the voids 11 is not particularly limited and can be appropriately set according to a size of the bone substitute 1. For example, when the size of the bone substitute 1 is large, the average diameter of the voids 11 may be about 10 mm

The average diameter of the voids 11 may be in a range of 200 μm to 500 μm. When the average diameter of the voids 11 is within the above-described range, the circulation of osteoblasts in the bone substitute 1 is promoted, the bone substitute 1 is easily replaced with new bone, and a treatment period of high tibial osteotomy (HTO) can be shortened.

Although the average diameter of the voids 11 has been described, the bone substitute 1 according to the embodiment is also characterized in that it has little variation in void diameters of the voids 11. When the bone substitute 1 has a small variation in the void diameters of the voids 11 and includes the voids 11 having a uniform size, and the bioactive glass layer is formed in the voids 11, a porosity and load resistance of the bone substitute 1 can be maintained at a high level.

When the porosity of the base material 10 is 65% or more and less than 90%, both a suitable amount of circulation of osteoblasts in the bone substitute 1 and a compression resistance strength can be achieved. When the porosity of the base material 10 is 90% or more, the load resistance of the bone substitute 1 is low, and it becomes difficult to withstand pressure during loading and use. When the porosity of the base material 10 is less than 65%, the amount of osteoblasts penetrating into the base material 10 decreases during use, the replacement speed to new bone decreases, and thus it is not preferable.

The bioactive glass layer is a layer which covers substantially the entire inner surface of the void 11.

The bioactive glass has an amorphous structure and is composed only of silicon, calcium, sodium, phosphorus, and oxygen. In the embodiment, the bioactive glass layer is formed of bioactive glass mainly composed of silicon dioxide, calcium oxide, sodium oxide, and diphosphorus pentoxide. The composition ratio of the bioactive glass is so-called 45S5 glass containing 45% by weight of silicon dioxide, 24.5% by weight of calcium oxide, 24.5% by weight of sodium oxide, and 6.0% by weight of diphosphorus pentoxide.

The inner surface of the void 11 is covered with the bioactive glass layer formed of the bioactive glass and having a thickness of less than 50 μm. When the thickness of the bioactive glass layer is less than 50 μm, the compression resistance strength of the base material 10 can be increased, and the performance with respect to penetration of osteoblasts into the base material 10 can be improved. In addition, the bioactive glass layer only needs to cover the entire inner surface of the void 11 for the purpose of increasing the compression resistance strength of the bone substitute 1, and a lower limit of a thickness thereof is not specifically limited. The thickness of the bioactive glass layer may be 10 μm or more and 20 μm or less. The bone substitute 1 can have a sufficient compression resistance strength and efficiency of production thereof can be increased by setting the thickness of the bioactive glass layer to 10 μm or more and 20 μm or less.

The average diameter of the voids 11 can be calculated by measuring the voids on an SEM image. The thickness of the bioactive glass layer can be measured from results of analyzing an SEM-EDX image.

The porosity of the base material 10 can be calculated by measuring an amount of β-TCP and the bioactive glass per unit volume of the bone substitute 1.

(Method for Producing Bone Substitute)

Next, a method for producing the bone substitute 1 according to the embodiment will be described.

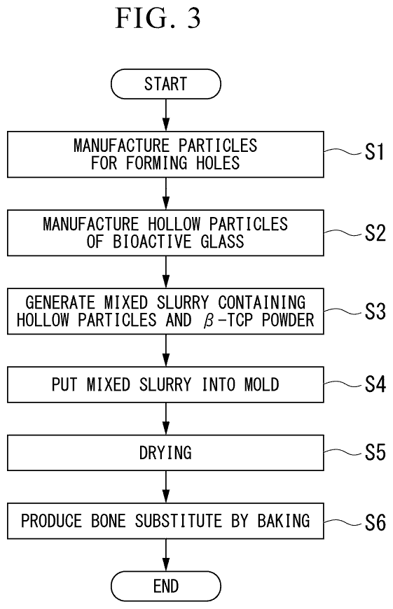

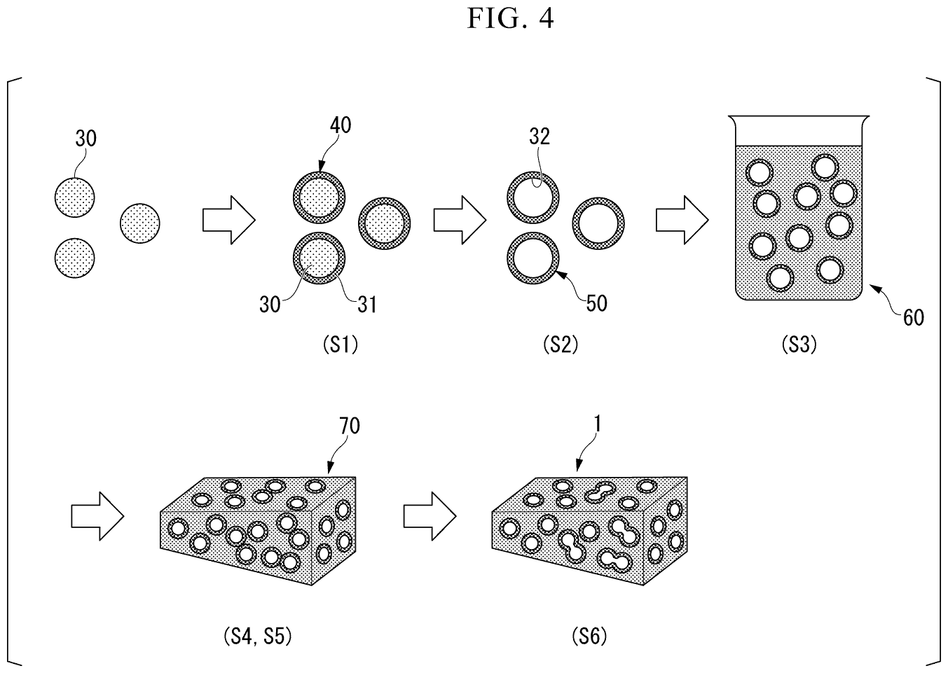

FIG. 3 shows a flowchart of the method for producing the bone substitute 1 according to the embodiment. FIG. 4 shows a schematic view of the method for producing the bone substitute according to the embodiment. As shown in FIGS. 3 and 4, in the method for producing the bone substitute 1, first, a surface of core particles for forming holes 30 is coated with a fine powder of the bioactive glass to form a glass coated layer 31, and thus particles for forming holes 40 are obtained (Step S1).

Beads formed of a polymer resin are used for the core particles 30. Since the polymer resin has high formability, core particles 30 having a shape close to a true sphere can be prepared. Further, when beads formed of a polymer resin are used, homogeneous core particles 30 with little variation in particle shape or particle diameter can be prepared.

Examples of the polymer resin constituting the core particles 30 includes methyl methacrylate, polymethyl methacrylate, polybutyl methacrylate, polyacrylic esters, polystyrene, and the like. When the core particles 30 formed of these resins are used, the glass coated layer 31 formed of the bioactive glass is sintered by baking, and the core particles 30 inside the glass coated layer 31 can be burned off (removed).

Spherical particles having an average particle diameter of 150 μm or more and 500 μm or less are used as the core particles 30. The core particles 30 can be appropriately selected according to a diameter of the voids 11 of the base material 10.

Further, in order to intentionally form a communication hole in which two or more voids 11 communicate with each other, for the core particle 30, aggregated particles may be added in addition to dispersed particles, and outer surfaces of the aggregated particles may be coated with the bioactive glass to form the glass coated layer 31.

A powder having an average particle diameter of less than 10 μm is used as a bioactive glass powder. The glass powder may be the above-described 45S5 glass. When the bioactive glass powder having an average particle diameter of less than 10 μm is used, the surfaces of the core particles can be uniformly coated with the bioactive glass powder.

In Step S1, the glass coated layer 31 is formed on the surface of the core particle by spraying the bioactive glass powder onto the surface of the core particle 30. A method for coating the core particles with the bioactive glass powder is performed by spraying a glass slurry containing the bioactive glass powder into a container while stirring the particles for forming holes with an air flow in the container, for example, using a rolling fluidized coating device. The glass slurry is prepared by dispersing the bioactive glass powder in a medium such as water, methyl alcohol, or ethyl alcohol. The glass slurry may contain a binding agent (a binder). The glass coated layer 31 can be uniformly formed on the surface of each of the core particles 30 while aggregation between the core particles 30 can be prevented using the rolling fluidized coating device. Further, when a concentration of the bioactive glass in the glass slurry is, for example, less than 10%, the core particles 30 are prevented from aggregating with each other, and the glass coated layer 31 can be uniformly formed on the surfaces of the core particles 30.

A thickness of the glass coated layer 31 can be adjusted according to an amount of glass slurry sprayed onto the core particles 30. In the case in which the thickness of the glass coated layer 31 is 50 μm or more, when it is baked together with a β-TCP powder, it becomes difficult for hollow particles 50 which will be described later to bind to each other. As a result, it is difficult for there to be communication between the voids 11. Further, in the case in which the thickness of the glass coated layer 31 is 50 μm or more, when it is baked together with the β-TCP powder, the sintering of the β-TCP powder is hindered, and strength of the base material 10 is lowered. The glass coated layer 31 may be 20 μm or less.

The glass coated layer 31 finally becomes the bioactive glass layer 20. As described above, the bioactive glass layer 20 only needs to cover the entire inner surface of the void 11 for the purpose of increasing the strength of the bone substitute 1, and the lower limit of the thickness is not particularly limited. However, in the manufacturing process, when the thickness of the glass coated layer 31 is too thin, the hollow particles 50 are likely to be damaged at the stage of manufacturing the hollow particles. Therefore, the thickness of the glass coated layer 31 may simply be 10 μm or more from a viewpoint of manufacturing. From the above, the thickness of the glass coated layer 31 is preferably less than 50 μm, and also when the thickness of the glass coated layer 31 is 10 μm or more and 20 μm or less, this is preferable because the manufacturing efficiency of the hollow particles 50 can be enhanced while the strength of the hollow particles 50 is maintained.

The thickness of the glass coated layer 31 is substantially equal to the thickness of the bioactive glass layer 20. In the bone substitute 1 which is the final product, the thickness of the bioactive glass layer 20 is preferably less than 50 μm and is more preferably 10 μm or more and 20 μm or less. Therefore, the thickness of the glass coated layer 31 in the particles for forming holes 40 is preferably 50 μm or less. When the thickness of the glass coated layer 31 is greater than 50 μm, it is difficult to form the communication hole which allows the voids 11 to communicate with each other, and the binding between the β-TCP powder particles of the base material 10 during the sintering which will be described later is hindered, and thus the strength of the base material 10 is lowered. On the other hand, when the glass coated layer 31 is too thin, this is not preferable because the hollow particles 50 are likely to be damaged when a mixed slurry is prepared or the like after the hollow particles 50 are produced in the next process. Considering these points, the thickness of the glass coated layer 31 is more preferably 10 μm or more and 20 μm or less.

Next, the particles for forming holes 40 are baked to obtain the hollow particles 50 (Step S2). The particles for forming holes 40 are heated at a temperature which is lower than a baking temperature in the process (Step S6) in which a sintered body which will be described later is manufactured and at which the polymer resin constituting the core particles 30 is burned off. As a result, the glass coated layer 31 is sintered, and the hollow particles 50 formed of the bioactive glass are obtained. Each of the hollow particles 50 has a strength such that a hollow shape thereof is maintained even in the mixed slurry in the next process.

Next, a mixed slurry 60 including the hollow particles 50 and the β-TCP powder is generated (Step S3). Specifically, the mixed slurry 60 is generated by dispersing the hollow particles 50 and the β-TCP powder in a medium formed of water or alcohol. When water or alcohol is used as the medium, a fluidity of the mixed slurry 60 is high, and it is easy to evaporate it by baking in the subsequent process (Step S6) in which the sintered body is manufactured. Moreover, even when a residue remains at the time of baking, it is harmless to a living body and is suitable as the bone substitute 1.

Since the hollow particles 50 define the porosity of the bone substitute 1, a mixing ratio between the β-TCP powder and the hollow particles 50 in the mixed slurry 60 is set by the porosity. For example, in the mixed slurry 60, the concentration of the β-TCP powder is preferably 40 to 60% by mass and the concentration of the hollow particles 50 is preferably 5 to 20% by mass.

Next, the mixed slurry 60 is put into a mold (Step S4). The mold has a wedge shape. In the mold, the hollow particles 50 are dispersed in the mixed slurry 60. The mixed slurry 60 put in the mold is dried (Step S5). The medium in the mixed slurry 60 is evaporated by drying, and a bulk body 70 in which the hollow particles 50 are dispersed in the aggregated β-TCP powder is molded.

The obtained bulk body 70 is put into a heating furnace and then heated and baked at a high temperature (Step S6). The sintered body obtained by sintering the β-TCP powder of the bulk body 70 by heating becomes the bone substitute 1. The bone substitute 1 has a wedge shape. In the obtained sintered body, the β-TCP powder and the bioactive glass of the hollow particles 50 are sintered, and a large number of voids are formed due to hollow portions 32 of the hollow particles 50. That is, in the mixed slurry 60, although the hollow particles 50 are dispersed in the β-TCP powder and the hollow particles 50 are voids, the bioactive glass of the hollow particles 50 and the β-TCP powder are sintered by the heating in Step S6, and the hollow portions 32 of the hollow particles 50 become the voids 11 of the bone substitute 1. Therefore, a void diameter of the voids 11 is substantially equal to the diameter of the core particle 30. In the voids 11, the β-TCP and the bioactive glass of the hollow particles 50 are in close contact with each other, and the bioactive glass layer 20 is formed on the inner surface of each of the voids 11.

In the bone substitute 1 of the embodiment, since the base material 10 has many spherical voids 11, the strength necessary for the bone substitute 1 can be maintained even when the base material 10 has a high porosity. Therefore, the bone substitute 1 can have sufficient compression resistance strength while promoting the replacement with new bone using the base material 10 having a high porosity.

In the bone substitute 1 of the embodiment, since the inner surface of the spherical void 11 is covered with the bioactive glass layer 20 having a thickness of less than 50 μm, the bioactive glass layer 20 serves as a strength member, and compressive strength of the base material 10 can be improved.

In the bone substitute 1 of the embodiment, since the thickness of the bioactive glass layer 20 is 10 μm or more and 20 μm or less, it is possible to maintain invasiveness of osteoblasts without reducing the porosity of the voids 11 while a function as a strength member is exhibited sufficiently.

In the bone substitute 1 of the embodiment, a content of the bioactive glass in a portion of the voids 11 other than the inner surface is less than 5% by mass when a mass of the bone substitute 1 is 100. That is, the bioactive glass which is the strength member can be unevenly distributed on the surfaces of the voids, and the compressive strength of the bone substitute 1 can be maintained at a high level. When the content of the bioactive glass in the portion of the void 11 other than the inner surface is less than 3% by mass, the compressive strength of the bone substitute 1 can be maintained at a higher level.

In the bone substitute 1 of the embodiment, since the average diameter of the voids 11 of the base material 10 is 150 μm or more, it is possible to secure an osteoblast flow path. Moreover, since the opening dimension L1 of the communication portion between the voids 11 can be set to 50 μm or more, osteoblasts can penetrate into a deep portion of the bone substitute 1, and the replacement with new bone can be promoted.

In the bone substitute 1 of the embodiment, since a plurality of communication holes through which the voids 11 communicate with each other are provided, osteoblasts can smoothly penetrate into the deep portion of the bone substitute 1. Therefore, the period during which the bone substitute 1 is replaced with new bone can be shortened.

In the bone substitute 1 of the embodiment, since the entire inner surface of the void 11 is covered with the bioactive glass layer 20 having a thickness of less than 50 μm, the compression resistance strength of the base material 10 can be maintained at a high level.

According to the method for producing the bone substitute according to the embodiment, since the particles for forming holes in which the glass coated layer 31 is formed on the core particles are manufactured, and then the hollow particles 50 in which the core particles 30 are burned off are put in the mixed slurry and dried and baked, the bone substitute in which the entire inner surface of each of the voids 11 is covered with the bioactive glass can be manufactured. Further, since the bioactive glass of the hollow particles 50 and the β-TCP powder are sintered, the bioactive glass layer 20 is reliably formed in the voids 11. Furthermore, since the particles for forming holes 40 are manufactured using the core particles 30 having a uniform particle shape and particle diameter, the voids 11 can have any shape and void diameter. Also, the porosity of the bone substitute 1 can be easily adjusted.

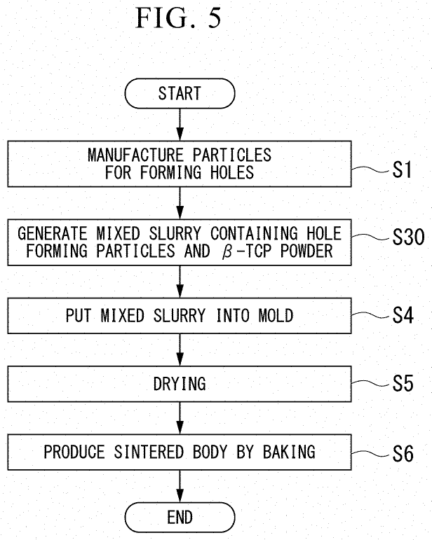

The method for manufacturing the bone substitute of the embodiment is not limited to the above-described method. FIG. 5 shows a flowchart of a modified example of the method for manufacturing the bone substitute 1. The above-described embodiment is an example in which Step S2 in which the hollow particles are manufactured is omitted.

First, the particles for forming holes 40 are manufactured by the same method as in the above-described embodiment (Step S1). Next, a mixed slurry containing the particles for forming holes 40 and the β-TCP powder is generated (Step S30). Specifically, a mixed slurry is generated by dispersing the particles for forming holes 40 and the β-TCP powder in a medium formed of water or alcohol. As the medium, the same medium as that in the above-described embodiment can be used.

A next process in which the mixed slurry is put into the mold (Step S4) and a process in which the mixed slurry put into the mold is dried (Step S5) are the same as those in the above-described modified example.

Next, the molded bulk body 70 is put into a heating furnace and heated and baked at a high temperature (Step S6). In the modified example, the core particles 30 of the particles for forming holes 40 are burned off by heating, and at the same time, the β-TCP powder is sintered. Therefore, in the case of the modified example, baking conditions in Step S6 are different from those in the above-described embodiment. In the modified example, the heating is firstly performed at a temperature at which the core particles 30 are burned. Thereafter, the heating is continued to sinter the β-TCP and the bioactive glass, and the obtained sintered body becomes the bone substitute 1. In the obtained sintered body, the β-TCP and the bioactive glass of the particles for forming holes 40 are sintered, the core particles 30 are removed, and thus a large number of voids 11 are formed. The void diameter is approximately equal to the diameter of the core particle 30. In each of the voids 11, the β-TCP and the bioactive glass are in close contact with each other, and the bioactive glass layer 20 is formed on the inner surface of each of the voids 11.

Also in the method for manufacturing the bone substitute according to the modified example, the bone substitute in which the entire inner surface of each of the voids 11 is covered with the bioactive glass can be manufactured, as in the above-described embodiment. Further, since the bioactive glass of the hollow particles 50 and the β-TCP powder are sintered, the bioactive glass layer 20 is reliably formed in the voids 11. Furthermore, since the particles for forming holes are manufactured using the core particles 30 having a uniform particle shape and particle diameter, the voids 11 can have any shape and void diameter. Moreover, the porosity of the bone substitute 1 can be easily adjusted.

As described above, although one embodiment of the present invention has been described, the technical scope of the present invention is not limited to the above-described embodiment, and it is possible to add various modifications to the elements, to delete the elements, or to combine the elements of the embodiments without departing from the spirit of the invention.

The shape of the base material 10 of the embodiment is not limited to the example which has the above-described wedge shape, and it can be set appropriately according to a shape of the bone defect part to be loaded into.

In the embodiment, the bone substitute 1 in which the spherical voids are formed using the core particles having a substantially true sphere shape has been described. However, according to the method for manufacturing the bone substitute according to the embodiment, since the core particles formed of a polymer are used, the shape of the voids can be variously changed.

Hereinafter, examples and comparative examples are shown below.

### EXAMPLES

Polystyrene beads having a particle diameter of 300 μm were prepared as the core particles. A bioactive glass powder (Bioglass (registered trademark) manufactured by Bonding Chemical company) having a particle diameter of 10 μm was dispersed in water to prepare a glass slurry having a bioactive glass concentration of 5% by mass. Core particles were put into the container of a rolling fluidized coating device, a glass slurry was sprayed into the container while stirring due to an air flow was performed, and thus particles for forming holes were obtained (Step S1). When the obtained particles for forming holes were measured with an SEM image, the thickness of the glass coating layer was 20 μm. The particles for forming holes were baked at 600° C. or higher, and hollow particles 50 were obtained (Step S2).

A β-TCP powder having an average particle size of 5 μm was prepared. Hollow particles, β-TCP powder, a deflocculant and water were mixed to obtain a mixed slurry (Step S3). A concentration of the hollow particles in the mixed slurry was 5% by mass, and a concentration of the β-TCP powder was 60% by mass. The mixed slurry was poured into a mold and dried in air to form a bulk body (Step S5). Thereafter, it was baked at 1000 to 1150° C. and sintered to obtain a bone substitute.

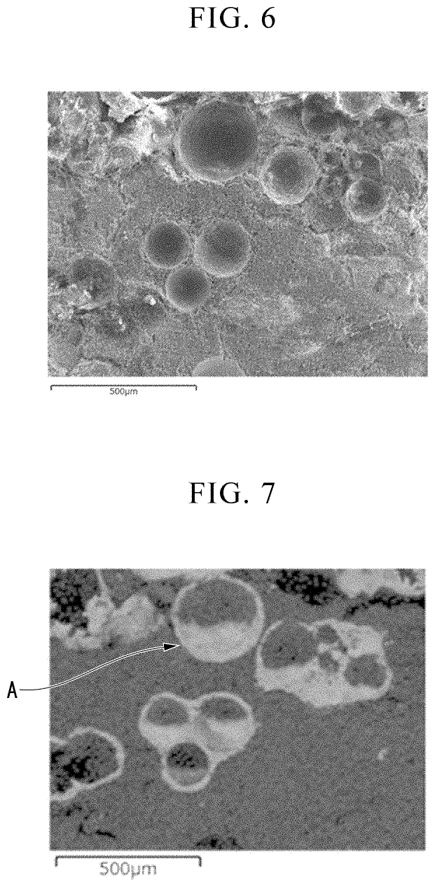

The SEM image of the bone substitute obtained in Example is shown in FIG. 6. Further, FIG. 7 shows an SEM-EDX analysis image of the bone substitute according to Example. As shown in FIGS. 6 and 7, a plurality of voids were formed and dispersed in the bone substitute of Example. As shown in FIG. 7, in the SEM-EDX analysis image, the distribution of the bioactive glass and the base material could be confirmed. As indicated by an arrow A in FIG. 7, a portion with high brightness is the bioactive glass. The result of the analysis by SEM-EDX was that it was confirmed that, in the bone substitute of Example, the bioactive glass layer was formed on the entire inner surface of each of the voids. The result of the measurement was that the average thickness of the bioactive glass layer was 20 μm. Further, within a range of 1×1 mm of the SEM-EDX image, no bioactive glass component was detected in the portion of the β-TCP other than the voids.

Many communication holes in which a plurality of voids communicated with each other were confirmed. The opening dimension L1 of the communication portion between the voids was 50 μm.

The compression resistance strength of the bone substitute obtained in Example was measured by an autograph, and it was 30 mph, and the strength desired by HTO was satisfied.

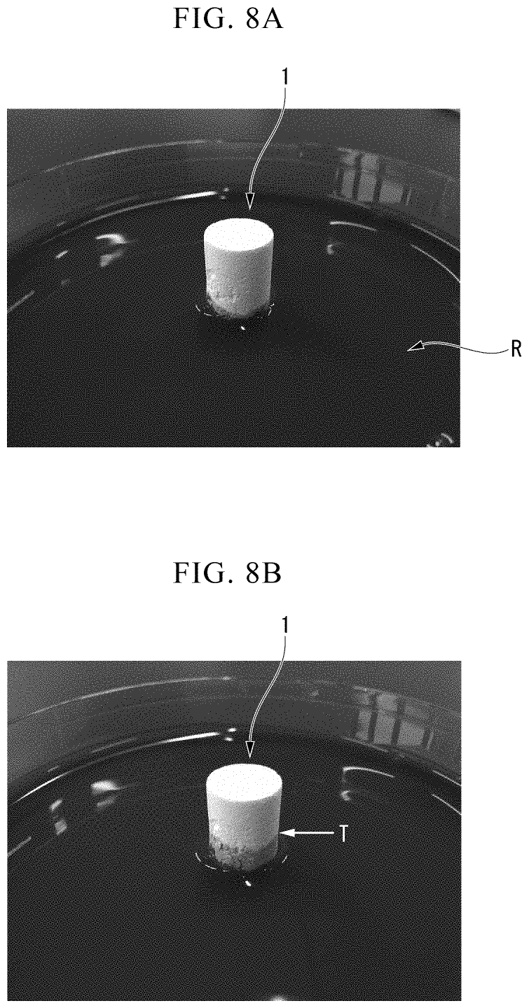

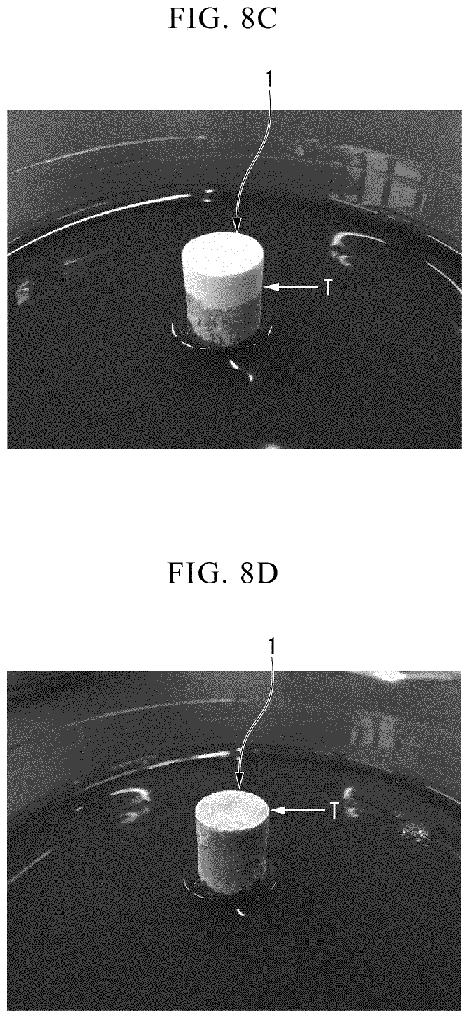

Moreover, a permeable performance test of a liquid into the bone substitute was performed with the following method. A rabbit blood R was prepared in a container to have a depth of about 1 mm The cylindrical bone substitute having a diameter of 10 μm and a height of 14 mm obtained in Example was placed in the container so that a circular surface was on the lower side, and a lower end portion of the bone substitute 1 was immersed in the rabbit blood R (refer to FIG. 8A). Since the bone substitute of the example was white, when the rabbit blood R permeated into the bone substitute 1, the bone substitute 1 was dyed red. Therefore, since a permeation state of the rabbit blood R into the bone substitute 1 could be visually confirmed, a change with time was visually observed.

FIGS. 8A to 8D show photographs of the changes with time. In FIGS. 8B to 8D, a boundary of a discolored portion of the bone substitute 1 is indicated by an arrow T. FIG. 8A is a photograph at the start of immersion. FIG. 8B is a photograph after 5 minutes from the start of immersion. After 5 minutes from the start of immersion, it was confirmed that the rabbit blood R had permeated to a height of about one quarter of the bone substitute 1. FIG. 8C is a photograph after 10 minutes from the start of immersion. After 10 minutes from the start of immersion, it was confirmed that the rabbit blood R had permeated to a center portion of the bone substitute 1 in a height direction. FIG. 8D is a photograph after 15 minutes from the start of immersion. After 15 minutes, it was confirmed that the rabbit blood R reached an upper end of the bone substitute 1 and permeated to the entire portion thereof. Thereafter, when the bone substitute 1 was cut and the inside of the bone substitute was visually observed, it was confirmed that the rabbit blood R had sufficiently permeated into the inside.

### Comparative Example

A fine powder of the bioactive glass having an average particle size of 10 μm was mixed with water in a container, and a glass slurry having a bioactive glass concentration of 10% by mass was prepared. A block-shaped porous β-TCP body having a porosity of 60% was prepared as the base material. The porous β-TCP body was immersed in the container in which the glass slurry was placed, and ultrasonic vibration was applied to the glass slurry in the container with a vibration device, so that the porous β-TCP body was impregnated with the glass slurry. The porous β-TCP body impregnated with the glass slurry was taken out of the container and dried in the air.

Next, the dried porous β-TCP body was put in a heating furnace and heated at 600° C. for 3 hours. The heated porous β-TCP body was cooled to solidify a film of the bioactive glass adhered to the inside the porous β-TCP body, and a bone substitute was obtained.

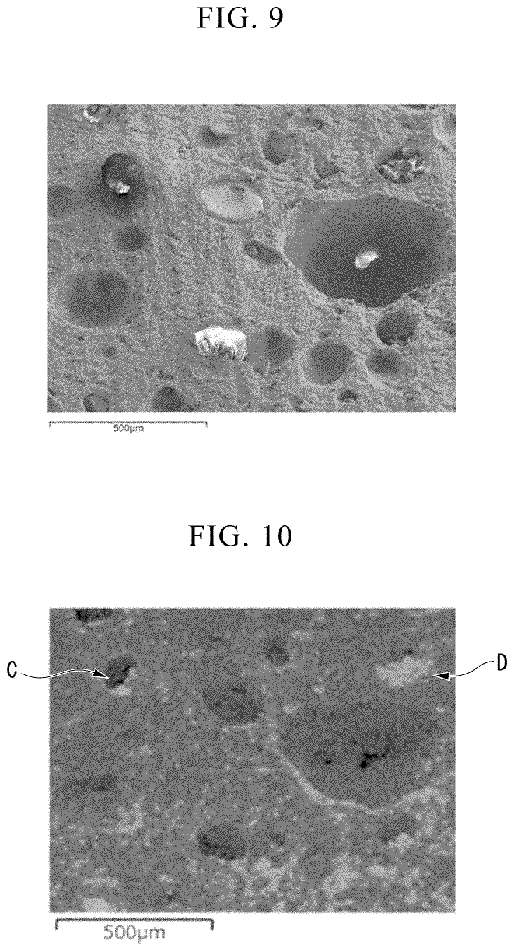

FIG. 9 shows an SEM image of the bone substitute obtained in the comparative example. A plurality of voids were formed in the obtained bone substitute, but there was a large variation in the size of the voids. Moreover, it was confirmed that the bioactive glass was present in a portion in which the voids were not formed.

FIG. 10 shows an SEM-EDX analysis image of the bone substitute of the comparative example. The bone substitute obtained in the comparative example was analyzed by SEM-EDX. As a result, although it was observed that the bioactive glass was adhered to the inner surfaces of the voids of the porous β-TCP body, there was a variation in an adhesion state of the bioactive glass, and there were a hole (refer to an arrow C shown in FIG. 10) in which the bioactive glass was adhered to only a part of the inner surface of the void and a portion (refer to an arrow D shown in FIG. 10) in which the bioactive glass was exposed outside the void. Moreover, the result of measuring the compression resistance strength of the bone substitute obtained in comparative example by an autograph was 20 mph which was inferior to the bone substitute of Example.

What is claimed is:

1. A bone substitute comprising:
a calcium phosphate-type porous ceramic body having spherical voids; and
a bioactive glass layer that is formed of a bioactive glass, coats an inner surface of each of the voids and has a thickness of less than 50 μm,
wherein the porous body has communication holes through which the voids communicate with each other.

2. The bone substitute according to claim 1, wherein the thickness of the bioactive glass layer is 10 μm or more and 20 μm or less.

3. The bone substitute according to claim 1, wherein, in the calcium phosphate-type porous ceramic body, a content of the bioactive glass in a portion of each of the voids other than the inner surface is less than 5% by mass when a mass of the bone substitute is 100.

4. The bone substitute according to claim 1, wherein an average diameter of the voids of the calcium phosphate-type porous ceramic body is 150 μm or more.

5. The bone substitute according to claim 1, wherein an average diameter of the voids of the calcium phosphate-type porous ceramic body is 200 μm or more and 500 μm or less.

6. The bone substitute according to claim 1, wherein a porosity of the calcium phosphate-type porous ceramic body is 65% or more and less than 90%.

7. The bone substitute according to claim 1, wherein:
the bioactive glass includes silicon dioxide, calcium oxide, sodium oxide, and diphosphorus pentoxide, and
a composition ratio of the bioactive glass is 45% by weight of silicon dioxide, 24.5% by weight of calcium oxide, 24.5% by weight of sodium oxide, and 6.0% by weight of diphosphorus pentoxide.

8. The bone substitute according to claim 1, wherein the communication holes have two or more of the voids communicate with each other, and the communication holes have a communication portion between the voids which has an opening dimension of 50 μm or more.

9. A method for producing the bone substitute including a calcium phosphate-type porous ceramic body having spherical voids and a bioactive glass layer which is formed of a bioactive glass, coats an inner surface of each of the voids, the method comprising steps of:
obtaining particles for forming holes by coating a fine powder of the bioactive glass on surfaces of core particles for forming holes;
obtaining hollow particles of the bioactive glass by baking the particles for forming holes to remove the core particles;
molding a bulk body from a slurry containing the hollow particles and calcium phosphate-type ceramic powder; and
obtaining the bone substitute by baking the bulk body.

10. A method for producing the bone substitute including a calcium phosphate-type porous ceramic body having spherical voids and a bioactive glass layer that is formed of a bioactive glass, coats an inner surface of each of the voids, the method comprising steps of:
obtaining particles for forming holes by coating fine powder of the bioactive glass on surfaces of core particles for forming holes;
molding a bulk body from a slurry containing the particles for forming holes and calcium phosphate-type ceramic powder; and
obtaining the bone substitute by baking the bulk body to remove the core particles.

11. The method according to claim 9, wherein the core particles have a spherical shape.

12. The method according to claim 9, wherein the core particles are formed of a polymer.

13. The method according to claim 10, wherein the core particles have a spherical shape.

14. The method according to claim 10, wherein the core particles are formed of a polymer.
